(19) United States
(12) Patent Application Publication
(10) Pub. No.: US 2012/0288819 A1
(43) Pub. Date: Nov. 15, 2012

(54) DENTAL IMAGING SYSTEM WITH ORIENTATION DETECTOR

(75) Inventors: E. Ronald Burrell, Healdsburg, CA (US); O. Rose Burrell, Healdsburg, CA (US)

(73) Assignee: E. Ron Burrell Dental Corporation, Santa Rosa, CA (US)

(21) Appl. No.: 13/103,255

(22) Filed: May 9, 2011

(51) Int. Cl. A61B 6/14

(52) U.S. Cl. 433/29; 433/215

(57) ABSTRACT

This disclosure provides systems, methods, and apparatus for dental imaging. In one aspect, a hand piece, having a lens for capturing light for an image sensor, is configured for obtaining images of teeth in a mouth. The hand piece is provided with an orientation sensor, such as a gyroscope. The orientation sensor detects the roll, pitch, and yaw of the hand piece. In operation, an image of a tooth is captured at a reference position, and the roll, pitch, and yaw of the hand piece is determined at that position. The hand piece is moved to another position, and additional images of teeth in the mouth are not obtained until the orientation of the hand piece at the other position matches the orientation of the hand piece at the reference position. The reference and second images may be combined, and the similar orientations of the hand piece at the reference and second positions can facilitate the combination by ensuring that the reference and second images are obtained from similar perspectives.

### TECHNICAL FIELD

This disclosure relates to dental imaging systems, particularly systems for obtaining multiple images of intra-oral features.

### DESCRIPTION OF THE RELATED TECHNOLOGY

Images of teeth in a mouth provide a basis for many aspects of modern dentistry. The images, which may also be referred to as intra-oral images, may be used for various purposes, including for diagnosing and detecting dental conditions. In addition, intra-oral images may be used in the fabrication of dental fixtures and prostheses. For example, images of a tooth may be captured to generate a three-dimensional model based on which a dental crown may be formed.

The accuracy of a diagnosis of a dental condition, or the fit and appropriateness of a dental fixture or prostheses as a replacement for, e.g., a tooth, depend on the accuracy of the images of the dental features being evaluated or replaced. Consequently, a continuing need exists to provide accurate intra-oral images.

### SUMMARY

One aspect of the subject matter described in this disclosure can be implemented in a dental imaging system. The dental imaging system can include an image sensor configured to capture an image of a tooth in a mouth. A hand piece has a light input aperture configured to capture and provide light to the image sensor. The hand piece is configured to fit and be movable within the mouth. An orientation detector is provided and configured to determine an orientation of the hand piece. A processor is electrically connected to the orientation detector and the image sensor. The processor is programmed to detect an orientation signal from the orientation detector and to control the image sensor based upon the orientation signal. The processor may also be programmed to store the reference orientation and compare subsequent orientations of the hand piece to the reference orientation.

In another aspect, a hand piece for dental imaging is provided. The hand piece includes a housing configured to fit and be movable within a mouth. The hand piece includes a light input aperture on the hand piece. The light input aperture is configured to capture and provide light to an image sensor configured to capture an image of a tooth in the mouth. An orientation detector is provided and configured to detect two or more of the roll, pitch, and yaw of the hand piece.

In yet another aspect, a method for capturing images of teeth in a mouth is provided. The method includes inserting a hand piece into the mouth; obtaining a reference image of a tooth at a reference position in the mouth; using a processor to determine a reference orientation of the hand piece, wherein the reference orientation is the orientation of the hand piece at the time of capturing the reference image; using the processor to subsequently determining an orientation of the hand piece before obtaining another image of one or more teeth in the mouth; and obtaining the other image when the hand piece is in the reference orientation. In some implementations, the other image is obtained only if the hand piece is in an orientation that matches the references orientation, such that two or more of the roll, pitch, and yaw of the hand piece in the position for taking the other image is substantially the same as the roll, pitch, and yaw of the hand piece at the reference orientation. For example, the processor may be programmed to disallow image capture unless the orientation of the hand piece substantially matches the reference orientation.

### BRIEF DESCRIPTION OF THE DRAWINGS

FIG. 1A illustrates an example of a schematic side view of a hand piece for obtaining intra-oral images.

FIG. 1B illustrates a schematic example of the information, such as roll, pitch, and yaw, obtained by an orientation sensor attached to the hand piece.

FIG. 2 illustrates schematically an example of an imaging system including the hand piece of FIGS. 1A and 1B.

FIG. 3A illustrates schematically an example of a top-down view of the hand piece of FIGS. 1A and 1B in position for obtaining an image of the occlusal surface of a tooth.

FIG. 3B illustrates schematically an example of a top-down view of the hand piece of FIGS. 1A and 1B in position for obtaining another image of the occlusal surface of a tooth.

FIG. 4 illustrates schematically an example of a top-down view of the hand piece of FIGS. 1A and 1B in position for obtaining an image of the side of a tooth.

FIG. 5 illustrates schematically an example of a tooth in isolation, viewed from the front of a mouth, along with various positions for the hand piece of FIGS. 1A and 1B to obtain images of the occlusal, buccal, and lingual surfaces of the tooth.

### DETAILED DESCRIPTION

Dental imaging systems may be used to obtain images of intra-oral features to facilitate fabrication of, for example, dental prosthesis such as dental crowns. For example, the dental imaging systems may include a hand piece that has a camera and that may be inserted into a mouth. Multiple images of an intra-oral feature may be obtained and these images may be combined to generate a three-dimensional model of the feature, for example, a tooth for which a crown will be made. A dental prosthesis may then be fabricated by computer aided manufacturing using the three-dimensional model to guide the fabrication process.

In many instances, a set that includes multiple images of a tooth are obtained to generate the three-dimensional model. These images may be taken from different positions and combined together by a computer. For example, a top-down image of a tooth may be captured and additional images of the tooth from its sides may be obtained. In addition, images of other teeth, e.g., neighboring teeth, may be captured to provide additional information regarding the shape and dimensions of the tooth and how the tooth interfaces with other teeth. These various images form a set from which a model is made and the model may be used to guide the fabrication of a dental crown.

It has been found that such a set of images can produce inaccurate models of intra-oral structures. In operation, the hand piece for obtaining the images may be moved into different positions to take these other images and, in these different positions, the hand piece may be at a slightly different orientation relative to the object being imaged. For example, for obtaining top-down views of different teeth, the hand piece may be horizontally translated into other positions. However, in addition to horizontally translating the hand piece, operator error may occur and the hand piece may also be inadvertently rotated or tilted. As a result, the orientation of the hand piece relative to the object being imaged may vary from position to position. For example, at the various positions, the hand piece may be tilted at slightly different angles relative to the object being imaged; in one position, for one image, the hand piece may be tilted towards the object being imaged and, in another position, for another image, the hand piece may be tilted away from the object being imaged. Due to the different perspectives, a feature may look larger or smaller in one image than in another image. In addition, because the images are taken from different perspectives, it can be difficult to establish a common baseline for evaluating the relative sizes and positions of features. Consequently, when the various images are combined, the perspectives of the various images may not match and the model of the intra-oral structure may be inaccurate. As a result, dental prosthesis formed using these images as a guide may be inaccurately proportioned. These prosthesis may not fit properly, leading to discomfort for the patient and/or increased expense and time to prepare a suitable prosthesis due to the need to fabricate a replacement prosthesis or rework an existing prosthesis.

Some implementations described herein provide systems, methods, and apparatus for providing highly accurate sets of dental images. In some implementations, the imaging system includes a hand piece that has an aperture for capturing light and that is configured to direct the light to an image sensor. The aperture can include, for example, a lens. The hand piece is provided with an orientation sensor, such as a gyroscope, which is connected to a computer system. The orientation sensor is configured to detect the roll, pitch, and yaw of the hand piece.

In some implementations, in operation, an image of a tooth is captured at a reference position, and the roll, pitch, and yaw of the hand piece at the moment of image capture are determined. This roll, pitch, and yaw information provides a reference orientation that can be stored, e.g., by being saved to a memory device, and subsequently used to compare the orientation of other images. For example, one or more subsequent images, with the hand piece at a different or at the same position, are later captured. The roll, pitch, and yaw of the hand piece for obtaining these images is determined. In some implementations, the system is programmed to prevent or disallow capture of the subsequent images until the roll, pitch, and yaw of the hand piece matches the reference orientation. In some other implementations, less than all (e.g., two) of the parameters of roll, pitch, and yaw are detected and/or evaluated to determine whether a given orientation matches the reference orientation.

In some other implementations, the images are captured, but the system tracks the orientation of the hand piece for each image and uses the orientation information to guide the combination of the various images. For example, the system may be set to simply disregard images taken in orientations that do not match the reference orientation. In such cases, multiple images are taken at each position to ensure that at least one of the images matches the reference orientation. In another example, the orientation information associated with each image may be stored and this orientation may be factored in during the combination process, and images that are taken from orientations that do not match the reference orientation are still used in the combination. Because the orientation of the hand piece for each image is known, the system may be configured to account for differences in perspective when combining the images together.

Because the orientations of the various images are known and may be made or required to match, aberrations caused by obtaining images from different perspectives or angles may be accounted for or avoided. As a result, a more accurate model or representation of a natural, intra-oral structure (e.g., tooth) may be obtained. The model may be used as a model from which a highly accurate dental prosthesis may be formed.

Reference will now be made to the Figures, wherein like numerals refer to like parts throughout. It will be appreciated that the Figures are not necessarily drawn to scale.

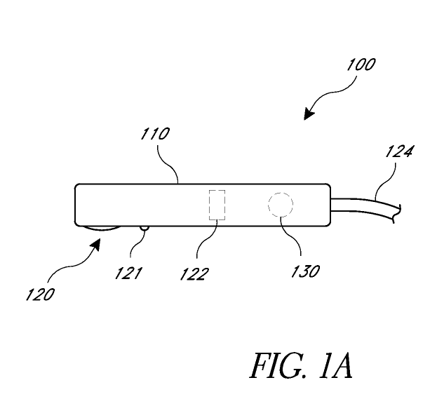

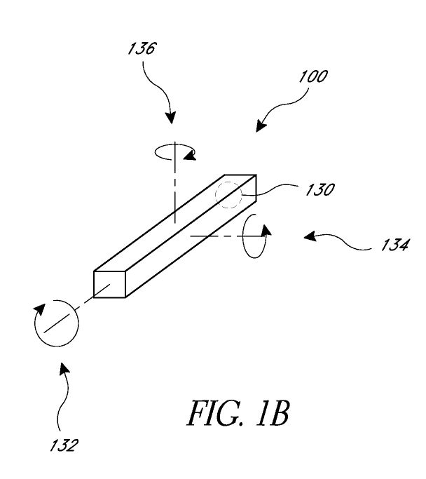

FIG. 1A illustrates an example of a schematic side view of a hand piece 100 for obtaining intra-oral images, in accordance with some implementations of the disclosure herein. The hand piece 100 has a housing 110 with a light input aperture 120. The light input aperture 120 may be a lens structure that captures light from an object (not shown) to be imaged. A light emitter 121 may be provided to illuminate the object in some implementations. The light input aperture 120 may be configured to capture light and direct the light to an image sensor 122. For example, the light may be directed from the light input aperture 120 to the image sensor 122 by optics and/or light guide structures (not shown) internal to the housing 110. The optical information provided by this light can be captured by the image sensor 122 to, e.g., obtain on image of an object. In some implementations, the light captured by the image sensor 122 is predominantly provided by the light emitter 121 and light from other sources, e.g., ambient light, is eliminated or kept at a sufficiently low level to prevent interference with image capture by the image sensor 122. In some implementations, the hand piece 100 is connected to a computer system (not shown) by an interconnect 124 and this information is electrically transmitted through the interconnect 124 to the computer system, where the information, such as an image, may be stored.

The image sensor 122 may be any suitable sensor that allows optical information to be converted to an electrical signal. Examples of suitable image sensors include charged-coupled device (CCD) and complementary metal-oxide-semiconductor (CMOS) image sensors.

With continued reference to FIG. 1A, the hand piece 100 includes an orientation sensor 130. The orientation may be any suitable sensor that allows the orientation of the hand piece 100 to be detected. In some implementations, the orientation sensor 130 detects the roll, pitch, and yaw of the hand piece 100. In some implementations, the sensor 130 is motion sensor. An example of a suitable orientation sensor is a gyroscope. The gyroscope may be digital gyroscope, which has advantages for providing a compact device that can be easily accommodated in the hand piece 100.

The orientation sensor 130 may be accommodated inside the housing 110 in some implementations. In some other implementations, the orientation sensor 130 may be attached to the hand piece 100, but may be disposed outside of the housing 110. For example, in some implementations, the orientation sensor 130 may be affixed to the housing 110 as a retrofit part to hand pieces that did not originally have such a sensor.

FIG. 1B illustrates a schematic example of the information obtained by the orientation sensor. As discussed herein, the orientation sensor 130 allows the roll, pitch, and yaw of the hand piece 100 to be detected. The roll parameter corresponds to the angle of rotation of the hand piece 100 about axis 132, which is the axis extending along the length of the hand piece 100. The pitch parameter corresponds to the angle of rotation of the hand piece 100 about axis 134, which is the axis extending perpendicular to the axis 132 on the same generally horizontal plane as the axis 132. The yaw parameter corresponds to the angle of rotation of the hand piece 100 about axis 136, which is the axis extending normal to the plane defined by the axis 132 and 136. In some implementations, relative to a three-dimensional Cartesian coordinate system, the axis 132 may be considered to correspond to the y-axis, the axis 134 may correspond to the x-axis, and the axis 136 may correspond to the z-axis, with the hand piece 100 centered at the origin of the coordinate system.

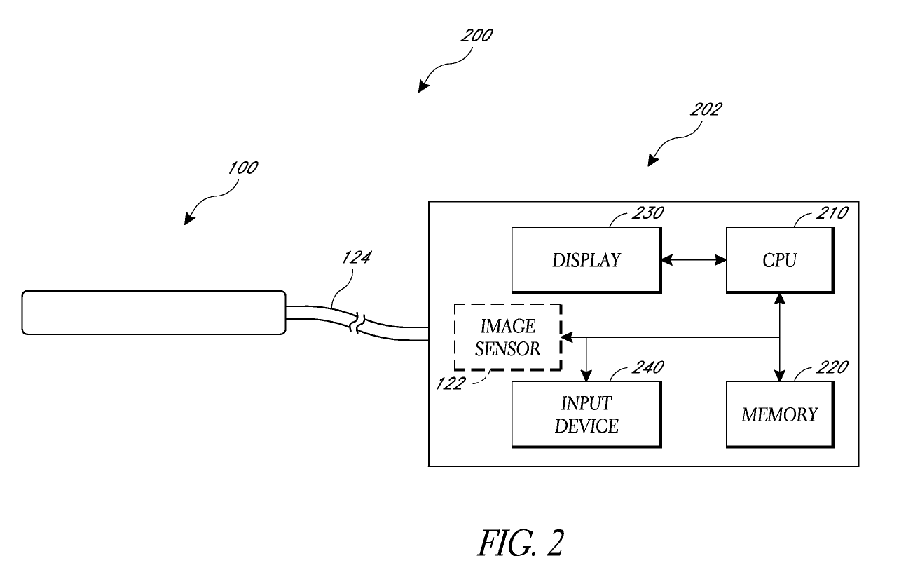

With reference now to FIG. 2, an example of an imaging system 200 including the hand piece 100 is illustrated schematically. The system 200 includes the hand piece 100, which is connected to a computer system 202. The hand piece 100 may be connected to the computer system 202 by a physical interconnect 124, which can include electrical and/or optical cabling. In some implementations, the hand piece 100 is “wirelessly” connected to the computer system 202. For example, the hand piece 100 may communicate with the computer system 202 using electromagnetic radiation. In such implementations, each of the hand piece 100 and the computer system 202 may be provided with a transmitter (not shown) and a receiver (not shown), which transmit and receive electromagnetic radiation, respectively. A wireless connection may be beneficial in some applications, as it allows movement of the hand piece 100 without the encumbrance of a wired connection.

With continued reference to FIG. 2, the computer system 202 includes a processor 210, e.g., a central processing unit (CPU), that is configured to execute computer programming. The system 202 may also include a memory 220, a display 230, and an input device 240, each of which may be configured to communicate with the processor 210. In some implementations, one or more of the memory 220, display 230, and input device 240 may be omitted or integrated together with one another or with the processor 210.

The computer programming for the system 202 may be stored or resident in the memory 220. The programming may include any code or instructions to perform any of the functions and actions discussed herein. The memory 220 may also be utilized to store orientation data and image or optical information from the hand piece 100. The memory 220 may take various forms, including volatile and/or non-volatile memory. In some non-limiting examples, the memory 220 may include one or more of random access memory (RAM), flash memory, magnetic memory devices, and firmware.

An operator may interact with the computer system 202 via the display 230 and the input device 240. The display 230 may include any device for visually presenting information to an operator. For example, the display can be a liquid crystal display (LCD) device or cathode ray tube (CRT) device. Information regarding the status of the system and the imaging procedure may be provided in the display 230. For example, the display 230 can show the view from the light input aperture 120 of the hand piece 100 (FIG. 1A) and indicate to the operator whether the orientation of the hand piece 100 matches the reference orientation. The operator may provide instructions or inputs to the system 200 using the input device 240. The input device 240 may be one or more various devices that can receive instructions or inputs from an operator and convert those instructions or inputs to electrical signals for transmission to other devices or modules in the computer system 202. For example, the input device 240 may include one or more of a keyboard, a button, a switch, a touch pad, a touch screen, a mouse, or a microphone for receiving voice commands.

With continued reference to FIG. 2, in some implementations, the image sensor 122 may be accommodated outside of the housing 110. For example, the image sensor 122 may be spaced apart from the housing 110 and connected to the hand piece 100 by the interconnect 124, which may be an optical interconnect in addition to being an electrical interconnect. The interconnect 124 can include optically transmissive material, e.g., a fiber optic cable, that allows light to propagate from the light input aperture 120 to the image sensor 122. In such implementations, the image sensor 122 may be accommodated as part of the computer system 202.

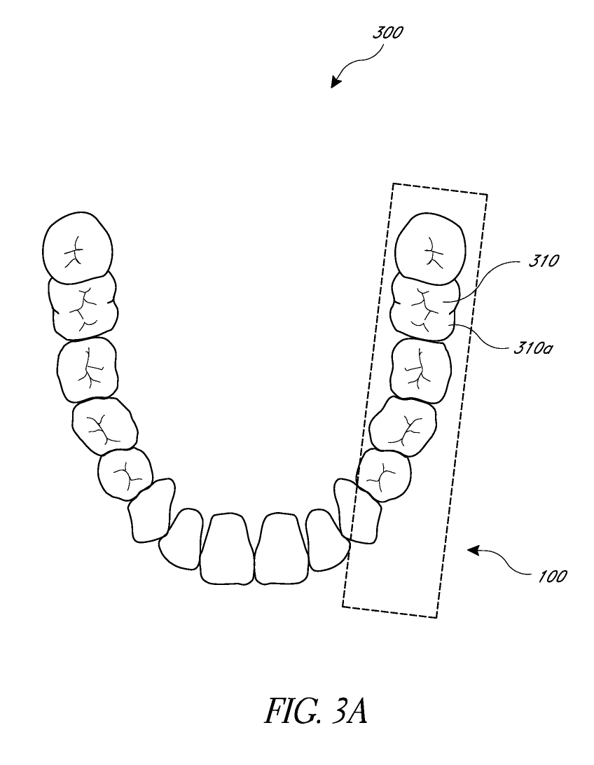

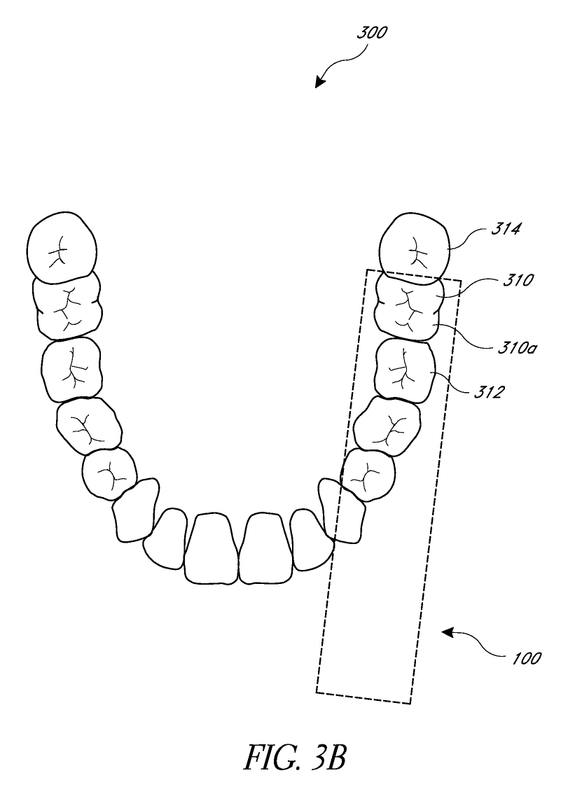

In operation, the image sensor 122 captures an intra-oral image when the hand piece 100 is positioned inside a mouth 300. FIG. 3A illustrates an example of a top-down view of the hand piece 100 in position for obtaining an image of the occlusal surface of a tooth. The hand piece 100 is positioned over a tooth 310 to obtain an image of occlusal surface 310a of that tooth and optionally the occlusal surface of neighboring teeth. In operation, the view of the tooth 310 from the light input aperture 120 (FIG. 1A) may be shown on the display 230 (FIG. 2). Upon seeing a desired view of the tooth 310, the operator can instruct the computer system 202 to obtain an image of the tooth 310. Using the orientation sensor 130 (FIG. 1A), the computer system 202 also registers the orientation of the hand piece 100 upon obtaining an image. For example, the roll, pitch, and yaw of the hand piece 100 at the time of obtaining the image can be recorded. This roll, pitch, and yaw may be used as a reference orientation for subsequent images.

Multiple views of the tooth 310 from different locations may be obtained to construct a three-dimensional model of the tooth 310 and determine how the tooth 310 interfaces with other features, e.g., other teeth, in the mouth 300. For example, images of neighboring teeth may be captured. FIG. 3B illustrates an example of a top-down view of the hand piece 100 in position for obtaining another image of the occlusal surface of a tooth. The hand piece 100 has been laterally translated and moved forward, towards the front of the mouth 300, relative to its position in FIG. 3B. In this position, a more direct view of neighboring tooth 312 may be obtained, while also providing a view of the surfaces of the tooth 310 that contact the tooth 312 (e.g., the mesial surface of the tooth 310). Similarly, the hand piece 100 may be horizontally translated towards the back of the mouth 300 to obtain an image of the tooth 314 and the distal surface of the tooth 310.

With continued reference to FIG. 3B, the orientation of the hand piece 100 at its new position is determined by the orientation sensor 130 (FIG. 1A). In some implementations, the computer system 202 may be programmed to automatically obtain an image of the tooth 312 once the orientation of the hand piece 100 matches the reference orientation. Alternatively or additionally, the computer system 202 may be programmed to prevent the operator from obtaining and recording an image until the reference orientation is matched, at which point the system will permit the operator to obtain an image of the tooth 312. In some implementations, the computer system 202 may be programmed to obtain multiple images at each of various different positions and also record the orientation information for each image. In some implementations, the system 202 then selects a set of images for model generation, the set of images being images that having matching orientations. Where multiple images are obtained from a particular position, some of the images may more closely match the reference orientation than other of the images, even if all the images are within the desired variance range from the reference orientation. In implementations where multiple images are obtained from a particular position, the system 202 may be programmed to select the image that most closely matches the reference orientation.

In some implementations, a given orientation may be considered by the system 202 to match the reference orientation when the roll, pitch, and yaw of the given orientation are each ± about 20°, ± about 10°, ±about 5°, or ± about 2° of the roll, pitch, and yaw of the reference orientation. In some implementations, the degree of variance from the reference orientation, and which is considered to be a match, may be operator selectable. The amount of acceptable variance to be considered a match for each of the roll, pitch, and yaw parameters may be the same, e.g., ± about 5°. In some implementations, the amount of acceptable variance to be considered a match for one or more of the roll, pitch, and yaw parameters may vary. For example, the variance may be ± about 5°for one of the parameters, the variance for one or more of the other parameters may be ± about 10° or ± about 2°.

In some implementations, only one or two of the roll, pitch, and yaw parameters may be gathered and evaluated to determine whether a given orientation matches the reference orientation. For example, only the roll and pitch of the hand piece 100 may be evaluated in some implementations. In such implementations, the yaw of the hand piece 100 may change while still being considered to the match the reference orientation. As seen from a top down view, one of ordinary skill in the art will appreciate that the yaw may change as the hand piece 100 is moved around the mouth 300 and tracks the curved placement of teeth in the mouth 300.

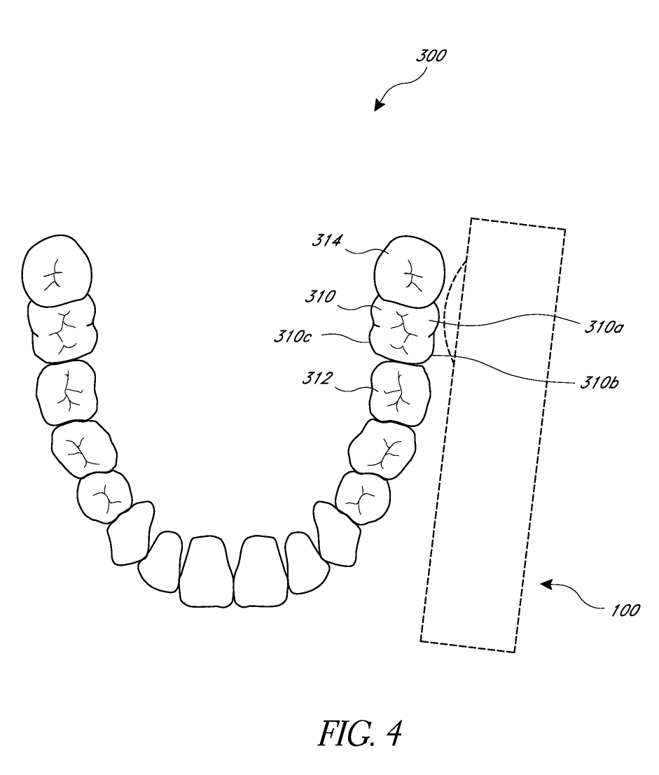

In addition to views of the occlusal surface of a subject tooth, side views of the tooth may be obtained. FIG. 4 illustrates an example of a top-down view of the hand piece 100 in position for obtaining an image of the side of a tooth. As illustrated, the hand piece 100 is positioned at the side of the tooth 310 to obtain an image of a buccal surface 310b of the tooth 310. In operation, the hand piece 100 may be moved to other positions towards the back or the front of the mouth 300 to obtain additional images of neighboring teeth (e.g., teeth 312 and 314) and additional views of the tooth 310. To ensure that the orientations of the hand piece 100 at each of these positions match one another, the orientation sensor 130 (FIG. 1) may be utilized to determine a reference orientation at a reference position. The reference orientations may then be used to determine whether the hand piece 100 is correctly oriented for obtaining additional images of the tooth 310 or other teeth in the mouth 300. As discussed herein, the orientation of the hand piece 100 at other positions is determined and, at the other positions, images are not obtained or used for modeling unless the orientation of the hand piece 100 at those other positions matches the reference orientation. Images of lingual surface 310c of the tooth 310 may be similarly obtained.

In some implementations, matching orientations may involve determining that a given orientation is the same as the reference orientation for all of the roll, pitch, and yaw parameters. As discussed herein, in some other implementations, matching orientations involves matching one or two of the roll, pitch, and yaw parameters. For example, as the hand piece 100 is moved around the curved placement of teeth in the mouth 300, the hand piece 100 may be expected to change yaw in some instances. In some implementations, only the roll and pitch of the hand piece 100 are evaluated to determine whether a given orientation matches the reference orientation.

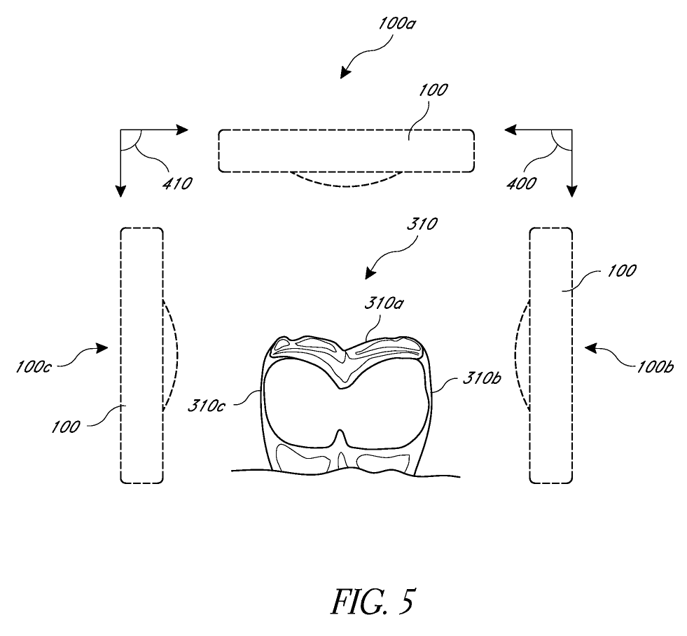

In some implementations, the reference orientations for obtaining occlusal, buccal, and/or lingual images may be linked. FIG. 5 illustrates an example of a view of the tooth 310 in isolation, along with the positions 100a, 100b, and 100c, of the hand piece 100 for obtaining images of the occlusal, buccal, and lingual surfaces 310a, 310b, and 310c, respectively, of the tooth 310. The hand piece 100 may be moved between the positions 100a, 100b, and 100c for obtaining images of the occlusal, buccal, and lingual surfaces 310a, 310b, and 310c, respectively. In the positions 100b and 100c, the hand piece 100 is rotated angles of 400 and 410, respectively, relative to the hand piece 100 at the position 100a.

In some implementations, one or both of the reference orientations at the positions 100b and 100c may be set by the operator independently of the reference orientation at the position 100c.

In some other implementations, one or both of the reference orientations at two of the positions 100a, 100b, and 100c may be set by reference to the other of those positions. For example, the positions 100b and 100c may be set by reference to the reference orientation at the position 100a. With continued reference to FIG. 5, one or both of the angles of rotation 400 and 410 of the hand piece 100 may be set at a predetermined value. For example, the angle of rotation 400 and/or 410 may correspond to the roll parameter of the hand piece 100 and may be set at, e.g., about 90°, or about 90°±10°, or about 90°±5°. The orientation sensor 130 (FIG. 1a) of the hand piece 100 determines the orientation of the hand piece at the positions 100b and 100c and the computer system 202 (FIG. 2) may the programmed to calculate the roll parameter for the reference orientation for the positions 100b and 100c. The system 202 may be programmed to deny the setting of orientations as reference orientations unless those orientations have a roll parameter that is equal to the calculated roll parameter. In some implementations, ensuring a particular amount of rotation in the roll parameter ensures that the hand piece 100 is sufficiently rotated to provide a view of the buccal or lingual surfaces 310b and 310c.

In some implementations, once a set of occlusal, buccual, and lingual images are obtained, the images may be combined to form a three-dimensional model. For example, the computer system 202 may be programmed to electronically stitch the various images together to generate the three-dimensional model. In some other implementations, the images may be transmitted to another computer system (not illustrated) apart from the computer system 202 and stitched together by that other computer system.

In some implementations, the three-dimensional model may be used to guide the fabrication of a dental prosthesis. Examples of prosthesis include dental crowns, ¾ crowns, inlays, onlays, and dental bridges. The three-dimensional model may be provided to a computer aided manufacturing system that uses the model to form a prosthesis of a desired shape, size, and composition. The matching orientations of the various images of the set provide a highly accurate three-dimensional model that can provide a dental prosthesis that provides a good fit within a mouth. Patient discomfort from poorly fitting prosthesis and/or the time and expense associated with modifying or refabricating the prosthesis can be reduced or avoided.

The various implementations disclosed herein may be modified in various ways apparent to those skilled in the art. For example, in some other implementations, the computer system 202 may be programmed to register orientation information for each image and use images to form a model even if the orientations of the images do not match. The system 202 may be programmed to use the orientation information to compensate for the slight differences in perspective of the images, thereby facilitating the generation of a highly accurate model of a subject feature, such as a tooth. In another example, while the hand piece 100 and related systems provide particular advantages when used in the fabrication of dental prosthesis, the hand piece 100 and related systems may also be utilized to provide a well-matched set of images in other applications, such as for diagnostic purposes.

Accordingly, it will also be appreciated by those skilled in the art that various omissions, additions and modifications may be made to the methods and structures described above without departing from the scope of the invention. All such modifications and changes are intended to fall within the scope of the invention, as defined by the appended claims.

What is claimed is:

1. A dental imaging system, comprising:
an image sensor configured to capture an image of a tooth in a mouth;
a hand piece having a light input aperture configured to capture and provide light to the image sensor, the hand piece configured to fit and be movable within the mouth;
an orientation detector configured to determine an orientation of the hand piece; and
a computer system electrically connected to the orientation detector and the image sensor, the computer system programmed to detect an orientation signal from the orientation detector and to control the image sensor based upon the orientation signal.

2. The dental imaging system of claim 1, wherein the orientation detector is a part of the hand piece.

3. The dental imaging system of claim 2, wherein the orientation detector is a gyroscope.

4. The dental imaging system of claim 2, wherein the orientation detector is configured to detect a roll, pitch, and yaw of the hand piece.

5. The dental imaging system of claim 4, wherein the computer system is programmed to store the roll, pitch, and yaw of the hand piece at a reference orientation, and is further programmed to compare subsequent orientations of the hand piece to the reference orientation.

6. The dental imaging system of claim 1, wherein the computer system is programmed to trigger image capture by the image sensor after determining an orientation of the hand piece in the mouth.

7. The dental imaging system of claim 6, wherein the computer system is programmed to trigger image capture when the orientation of the hand piece matches a reference orientation.

8. The dental imaging system of claim 7, wherein the computer system is programmed to determine the orientation of the hand piece at the time of image capture by the image sensor, wherein the reference orientation is the orientation of the image sensor during an earlier capture of an image.

9. The dental imaging system of claim 1, wherein the computer system is programmed to determine whether to combine one tooth image with another tooth image based upon the orientations of the hand piece when the tooth images were taken.

10. The dental imaging system of claim 1, wherein the computer system is programmed to align one tooth image with another tooth image based upon the orientations of the hand piece when the tooth images were taken.

11. The dental imaging system of claim 1, wherein the light input aperture comprises a lens.

12. The dental imaging system of claim 11, wherein the image sensor comprises a charged coupled device.

13. The dental imaging system of claim 1, further comprising a light source configured to output light from the hand piece an object to be imaged.

14. A hand piece for dental imaging, comprising:
a housing configured to fit and be movable within a mouth;
a light input aperture on the hand piece, the light input aperture configured to capture and provide light to an image sensor configured to capture an image of a tooth in the mouth; and
an orientation detector configured to detect two or more of a roll, pitch, and yaw of the hand piece.

15. The hand piece of claim 14, wherein the orientation detector comprises a motion detector.

16. The hand piece of claim 15, wherein the orientation detector is a gyroscope.

17. The hand piece of claim 14, wherein the light input aperture comprises a lens and the image sensor is disposed within the housing.

18. The hand piece of claim 14, wherein the orientation sensor and the image sensor are electrically connected to a computer system programmed to delay image capture by the image sensor until two or more of the roll, pitch, and yaw of the housing matches a predetermined reference orientation.

19. A method for capturing images of teeth in a mouth, the method comprising:
inserting a hand piece into the mouth;
obtaining a reference image of a tooth at a reference position in the mouth;
determining a reference orientation of the hand piece, wherein the reference orientation is the orientation of the hand piece at the time of capturing the reference image;
subsequently determining an orientation of the hand piece before obtaining another image of one or more teeth in the mouth; and
obtaining the other image when the hand piece is in the reference orientation.

20. The method of claim 19, wherein subsequently detecting the orientation is performed after moving the hand piece to another position within the mouth.

21. The method of claim 20, wherein subsequently detecting the orientation includes using an orientation detector and computer system to determine the orientation.

22. The method of claim 21, wherein the computer system is programmed to delay image capture of teeth at the other position until the orientation of the hand piece at the other position matches the reference orientation.

23. The method of claim 21, wherein the orientation of the hand piece at the other position matches the reference orientation when the roll, pitch, and yaw of the hand piece at the other position is about ±10° of each the roll, pitch, and yaw of the hand piece at the reference orientation.

24. The method of claim 23, wherein the orientation of the hand piece at the other position matches the reference orientation when the roll, pitch, and yaw of the hand piece at the other position is about ±5° of each the roll, pitch, and yaw of the hand piece at the reference orientation.

25. The method of claim 19, wherein the reference and second images comprise occlusal surfaces of teeth, further comprising obtaining additional images of buccal or lingual surfaces of the teeth.

26. The method of claim 25, wherein obtaining additional images of buccal or lingual surfaces of the teeth comprises:
using the computer system to determine orientations of the hand piece before taking the images of buccal or lingual surfaces of the teeth; and
obtaining the images of buccal or lingual surfaces of the teeth after the processor determines that a roll angle of the hand piece has been shifted by about 90° relative to the roll angle of the hand piece at the reference orientation.

27. The method of claim 25, wherein the roll angle during obtaining the images of the buccal or lingual surfaces of the teeth is within about 90°±5° of the roll angle of the hand piece at the reference orientation.

28. The method of claim 25, wherein obtaining the other image occurs before obtaining the additional images of buccal or lingual surfaces of the teeth.
